(19) United States
(12) Patent Application Publication
(10) Pub. No.: US 2015/0353982 A1
(43) Pub. Date: Dec. 10, 2015

(54) MICRO-EVOLUTION OF MICROBES

(71) Applicants: UNIVERSITY OF NEWCASTLE UPON TYNE, Tyne and Wear (GB); TANTILLUS SYNERGY LIMITED, Coventry (GB)

(72) Inventors: David Christian Daniel Van Alstyne, St. Cyrus (GB); Chelsea Marie Brain, Guildford (GB); Garyt Stephen Caldwell, Ashington (GB)

(21) Appl. No.: 14/758,196

(22) PCT Filed: Dec. 30, 2013
(86) PCT No.: PCT/GB2013/053436

§ 371 Date: Jun. 26, 2015

Related U.S. Application Data
(60) Provisional application No. 61/747,050, filed on Dec. 28, 2012.

(51) Int. Cl. C12Q 1/04; C02F 3/34; C02F 3/32; C12M 1/42; C12M 1/00

(52) U.S. Cl. CPC C12Q 1/04; C12M 35/08; C12M 35/00; C12M 29/00; C02F 3/322; C02F 3/34; C02F 2101/103

(57) ABSTRACT

An enclosed bioremediation system utilizing resistant micro-evolved microbes for treatment of wastewater and recovery of chemicals and metals and which also results in biomass and biochemical production and carbon capture.

The present application claims priority to U.S. Provisional Patent Application Ser. No. 61/747,050 entitled “Micro-evolution of Algal Strains”, filed 28 Dec. 2012 which is herein incorporated by reference in its entirety for all purposes.

### BACKGROUND

Micro-Evolution for Bioremediation and Recovery

Algae have the ability to adapt rapidly to a wide range of pollutants [1, 2]. There are two ways in which algae can change to cope better with the surrounding environment. Physiological acclimation (or natural/phenotypic plasticity) involves modifications in gene expression. However in more extreme conditions, where physiological acclimatization cannot provide tolerance, survival is dependent on adaptive microevolution, where random spontaneous genetic mutations confer resistance [3-5]. Here the mutation is pre-selective, and is not induced by the conditions. Microevolution can therefore change the genotype and phenotype of a whole algal population on a short time scale, over a small number of generations.

Bioremediation by microorganisms for biodegradation of pollutants is an environmentally safe, inexpensive and efficient decontamination technology [6]. Algae also has the ability to accumulate heavy metals by a number of different mechanism for removal of contaminants or recovery [4], which is a beneficial feature as heavy metal contaminated wastewaters or water sources near industrial areas is environmentally problematic. These waters can also contain high amounts of precious and expensive heavy metals of finite resource used in the electronics industry. These industries are looking for ways in which to clean-up these waters and recover particular heavy metals for reuse.

Although acclimation is a beneficial feature of algal cultures, selection of strains with resistance through pre-selective adaptation or microevolution will ensure stable culture for use in a bioremediation system with variable wastewaters. The use of microevolved algal and cyanobacterial strains for bioremediation and recovery activities as opposed to genetically modified organisms (GMOs) will also avoid environmental safety concerns and problematic regulations which may restrict use.

Mechanisms of Heavy Metal Tolerance

The requirement for metals Mn, Fe, Co, Ni, Cu, and Zn in trace amounts by microalgae and cyanobacteria has been widely studied [4]. These metals are important for biological metabolic activity such as electron transport and in the acquisition and assimilation of carbon, nitrogen and phosphates. However, in high concentration, these metals can be toxic to microalgae. Microalgae can adapt to high metal concentration through various phenotypic mechanisms. The physiological adaptation of microalgae through exposure can be demonstrated by the presence of algae in contaminated waters in present or past regions of heavy industry. Physiological and genetic mechanisms of tolerance exist, however it is difficult to distinguish by which method tolerance arose. Resistance is the preferred phrase used over tolerance when speaking of genetic adaptation. The common mechanisms of metal tolerance in algae as shown in FIG. 1 may be a result of the deployment of more than one of these mechanisms.

Extracellular Complexation and Uptake

The extracellular materials of algal cell walls/membranes can bind metal ions, reducing bioavailability. This lowers intracellular accumulation and decreases toxicity, conferring tolerance in a high metal concentration environment FIG. 1(3). The extracellular material in microalgae and cyanobacteria is primarily extracellular polysaccharide [7]. Microalgae have been shown to bind high value metals such as gold [8]. The presence of carboxyl, phosphatic and other negatively-charged groups on algal cell walls/membranes, involved in ion exchange, are also thought to be involved in regulating uptake and therefore bioavailability and toxicity tolerance FIG. 1(2), however the exact role is disputed [9]. Metals can also be chelated by metal-complexing ligands—siderophores—secreted from the cell, reducing bioavailability.

Intracellular Detoxification

Mechanisms of metal exclusion/efflux and reduced influx from the cell after uptake also enables tolerance in high metal concentration environments FIG. 1(1). Reduced influx can be regulated by alterations in permeability of the cell membrane to metal ions and possible modifications within the cell wall such as thickening for protection. Active transport/efflux of metal from within the cell can also allow tolerance to high environmental metal concentrations. Such systems have been widely reported in novel resistant bacterial strains [10].

Metal tolerance can also occur through sequestration of metals intercellularly through metallothioneins and phytochelatins [11, 12]. In addition, polyphosphate bodies accumulated in the cell under conditions of surplus phosphates are also intracellular sites for metal complexation [13]. The polyanionic properties of these phosphate polymers sequester cations; however, subsequent phosphate deficiency leading to mobilization of intercellular stored phosphate can lead to release of high toxic concentration of previously chelated metal ions within the cell.

There is therefore a need in the art to explore the feasibility and application of a process, allowing the selection of resistant strains whose resistance has arisen through the mechanism of microevolution, for the commercial production of resistant strains for use in multi-process wastewater treatment/recovery and production system.

### SUMMARY OF THE INVENTION

The invention provides a remediation system, the remediation system comprising a vessel and a microbial population having tolerance to a test composition. In a preferred embodiment the system is selected from the group consisting of a bioremediation and recovery system, a carbon capture system, a biochemical production system and a biomass production system.

In a preferred embodiment the microbial population is cultured from a microbe selected for biological remediating activity using a method for selecting a mutation in a gene or a change in a chemical modification of a gene that provides the microbe with the ability to be tolerant to the test composition. In a preferred embodiment, the mutation is an extant mutation. In another preferred embodiment, the change in a chemical modification of a gene is an extant change in a chemical modification of a gene. In one preferred embodiment the test composition is a heavy metal. In another preferred embodiment the test composition is a metal. In another preferred embodiment the test composition is an organic compound. In a more preferred embodiment, the remediation system comprises a microbe comprising biological activity, the biological activity comprising inactivating activity, whereby the inactivating activity inactivates, detoxifies, or degrades the test composition. In another more preferred embodiment, the remediation system is a carbon-capture system. In a yet other preferred embodiment, the remediation system is a wastewater treatment system. In a yet further preferred embodiment, the remediation system is a biomass production system. In another further preferred embodiment, the remediation system is a biochemical production system. In a most preferred embodiment, the remediation system is a bioremediation system.

In another embodiment, the invention provides a remediation system, the remediation system comprising a vessel, the vessel comprising the micro-evolved microbe, a fluid, a fluid input aperture, a fluid output aperture, and at least one gas input/output aperture. In a preferred embodiment the aperture comprises a valve. The valve may be a one-way valve, a two-way valve, or a multi-way valve. In one preferred embodiment the fluid is wastewater. In an alternative preferred embodiment the fluid is organic waste. In a yet further alternative embodiment the fluid is a hydrocarbon mixture.

The invention provides a micro-evolution (ME) process for creating a microbial population tolerant to a test composition, the micro-evolution process comprising the following steps: (i) providing a single microbe, the microbe isolated by serial dilution of a microbial population; (ii) providing a culture medium; (iii) adding the single microbe to the culture medium; (iv) providing the single microbe in the culture medium an environment whereby the single microbe replicates thereby creating daughter microbes; (v) treating the culture medium with a test composition whereby the test composition selects those daughter microbes having genetic resistance to the test composition; (vi) using fluctuation analysis to determine whether the selected daughter microbes comprise a change in gene expression (transcription and/or translation) or a change in a gene (mutation and/or chemical modification); (vii) selecting only those daughter microbes having a change in gene, wherein the change in gene is micro-evolution; the process resulting in a microbial population tolerant to the test composition.

The invention also provides a method for remediating a pollutant, the pollutant comprising a test composition, the method comprising the steps of: (i) providing a single microbe, the microbe isolated by serial dilution of a microbial population; (ii) providing a culture medium; (iii) adding the single microbe to the culture medium; (iv) providing the single microbe in the culture medium an environment whereby the single microbe replicates thereby creating daughter microbes; (v) treating the culture medium with a test composition whereby the test composition selects those daughter microbes having genetic resistance to the test composition; (vi) using fluctuation analysis to determine whether the selected daughter microbes comprise a change in gene expression (transcription and/or translation) or a change in a gene (mutation and/or chemical modification); (vii) selecting only those daughter microbes having a change in a gene, wherein the change in a gene is micro-evolution; the micro-evolution process resulting in a microbial population tolerant to the test composition; (viii) culturing the microbial population with the pollutant for a predetermined period of time; (ix) separating the microbial population from the pollutant, the method resulting in the remediation of the pollutant.

In one preferred embodiment the microbe is a microalga. In a preferred embodiment, the microalga is selected from the group consisting of Chlamydomonas, Chlorella, and Scenedesmus. In a more preferred embodiment, the microalga is Chlamydomonas nivalis. In another more preferred embodiment, the microalga is Chlorella vulagris. In another more preferred embodiment, the microalga is Scenedesmus dimorphus. In another preferred embodiment, the microbe is a cyanobacterium. In a preferred embodiment, the microalga is selected from the group consisting of Chlamydomonas, Chlorella, and Scenedesmus. In a more preferred embodiment, the microalga is Chlamydomonas nivalis. In another more preferred embodiment, the microalga is Chlorella vulagris. In another more preferred embodiment, the microalga is Scenedesmus dimorphus.

In another preferred embodiment, the microbe is a cyanobacterium. In a more preferred embodiment, the cyanobacterium is Arthrospira platensis.

In a yet other preferred embodiment, the microbe is a gram-positive bacterium. In a more preferred embodiment, the gram-positive bacterium is Bacillus subtilis.

In a preferred embodiment the test composition comprises a heavy metal selected from the group consisting of arsenic, cadmium, hexavalent chromium, lead, mercury, antimony, cobalt, copper, iron, manganese, molybdenum, nickel, selenium, silver, tin, vanadium, and zinc. In an alternative preferred embodiment, the test composition comprises an organic compound selected from the group consisting of organophosphates, organochlorines, carbamates, polycyclic aromatic hydrocarbons, polychlorinated biphenyls, triazines, phenoxy herbicides, dioxins, and alkaloids. In another alternative preferred embodiment, the test composition is a metal selected from the group consisting of aluminium, beryllium, and magnesium.

The invention also provides subjecting the microbe to an environmental stress but not to the test composition. In one preferred embodiment, the environmental stress is selected from the group consisting of a rapid change in temperature, change in alkalinity, change in dissolved carbon dioxide, change in dissolved oxygen, change in salinity, change in nutrient levels of the medium, change in the incident wavelength of light, and change in NH₄O₃ levels.

The invention also provides a carbon-capture process using the micro-evolution process disclosed herein, wherein further the microbial population fixes carbon dioxide from the environment and metabolically sequesters the carbon dioxide into a microbial biochemical. In one preferred embodiment, the microbial biochemical further is harvested and isolated from the microbial population.

The invention also provides use of the micro-evolution process disclosed herein, wherein the use is remediation. In one preferred embodiment, the remediation is bioremediation. In one more preferred embodiment, the bioremediation is selected from the group consisting of wastewater bioremediation, contaminated water bioremediation, hazardous waste bioremediation, and radioactive waste bioremediation.

The invention also provides use of the micro-evolution process disclosed herein, wherein the use is a carbon-capture system. In a yet other preferred embodiment, the remediation system is a wastewater treatment system. In a yet further preferred embodiment, the remediation system is a biomass production system. In another further preferred embodiment, the remediation system is a biochemical production system.

### BRIEF DESCRIPTION OF THE DRAWINGS

FIG. 1. Schematic of common mechanisms of metal tolerance in microalgae (adapted from Rai, 2001).

FIG. 2 illustrates a typical outline of the micro-evolution process: flow-diagram outlining the microevolution process to be used in the experiments. Final steps may also include testing metal uptake mechanisms and genetic sequence analysis to identify advantageous mutation. Dotted lines show dependent data-feedback loops.

FIG. 3. Schematic of the ME experimental layout (from Gonzales et al., 2012 [14]).

FIG. 4. A: Death curves and B: EC50 (non-linear regression curve) of A. platensis exposed to Cu²⁺ ions (mg/L).

FIG. 5. Microscope image comparisons showing morphological changes during exposure of A. platensis to high copper ion concentration with a and b: after 24 hour 1 mg/L Cu2+, c and d: Control after 24 hours 0.003 mg/L F/2 medium. High [Cu] has caused fragmentation of the trichomes. Cyanobacterium became light/bleached, segmented and prone to entanglement making them difficult to see/capture under a light microscope.

FIG. 6. Effect of (concentration 1 mg/L) Cu²⁺ ions on A. platensis culture. Note disintegration and clumping (left) and bacterial breakdown of cellular material (right).

FIG. 7. Selections for concentrations thereafter used for microevolution (ME) process.

FIG. 8. Box-plots for cell count distribution of ME sets for 0.3 (A) and 0.6 mg/L (B) Cu²⁺ ion concentration. Plots show the high variability of MO number in Set 1 of both 0.3 and 0.6 mg/L Cu²⁺ ion experimental concentrations, compared to control sets 2, as would be expected.

FIG. 9. Schematic comparing adaptive microevolution to the acclimation process.

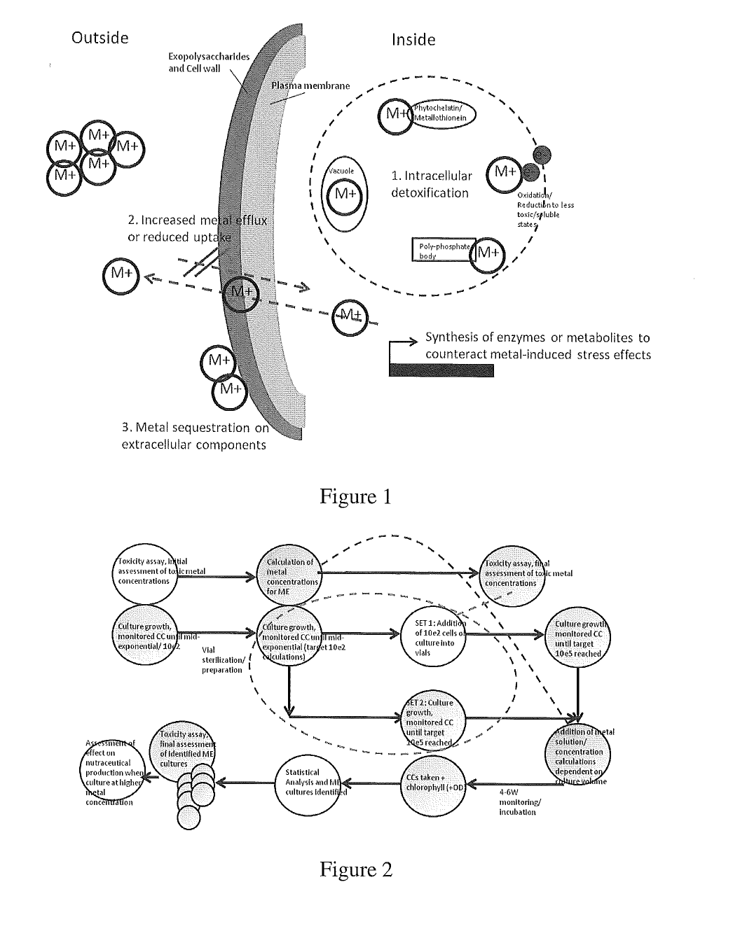

### DETAILED DESCRIPTION OF THE INVENTION

The invention encompasses a novel remediation system that utilizes chemical and/or metal (contaminant) resistant algae and/or cyanobacteria developed through micro-evolution. The resistant microorganisms are produced through a process of isolation and cloning, followed by exposure to varying concentrations of contaminant at commercially important or industrially problematic levels. Micro-evolved algal and/or cyanobacterial cultures are then selected through fluctuation analysis using models based on Luria-Delbruck (Luria, S., Delbruck, M. 1943. Mutations of bacteria from virus sensitivity to virus resistance. Genetics 28, 491-511) and comparison to Poisson distribution whereby micro-evolved cultures have arisen when variance (in culture density) is larger than the mean as opposed to variance equal to the mean as in Poisson distribution (see, for example, Gonzalez, R., et al., 2012. Adaptation of microalgae to lindane: A new approach for bioremediation. Aquatic Toxicology 109, 25-32 [14] for a similar method).

The invention also encompasses a novel wastewater bioremediation system. The invention also encompasses a novel carbon-capture system. The invention also encompasses a novel metal recovery bioremediation system. The invention also encompasses a novel heavy metal recovery bioremediation system. The invention also encompasses a novel biomass production system. The invention also encompasses a novel biochemical production system.

The system utilizing these micro-evolved alga and/or cyanobacteria (employing an enclosed tank or vessel and) has the ability to process and take-up contaminants for wastewater treatment/bioremediation (nitrate, phosphate, metals, and toxic chemicals) and subsequent discharge. The system may also allow recovery of certain contaminants (which may be high value finite resources such as metals, chemicals, phosphate) with possible synergistic production of biochemicals (biofuels or nutraceuticals [such as oils, pigments, and/or protein]) after processing. The system may also have the capability of carbon capture. The system avoids the use of genetically modified microorganism by means of direct gene insertion.

A benefit of the invention is that it enables a mutagen to aid mutation rate for next step/selection process, then conditions are altered (for example, stress) to select for strain with favorable mutation followed by next growth phase. Through adding stresses, such as exposure to high levels of contaminants or ultra violet light, strains are developed that are capable of performing specific biochemical functions that would not normally occur in nature.

In another embodiment the stress is environmental stress, such as, but not limited to, a rapid change in temperature, change in alkalinity, change in dissolved carbon dioxide, change in dissolved oxygen, change in salinity, change in nutrient levels of the medium, change in the incident wavelength of light, and change in NH₄O₃ levels.

In one embodiment a biorefinary model is presented employing the combined use of high heavy metal (or toxic chemical) wastewater as a feed stock for synergistic bioremediation/heavy metal recovery and nutraceutical/biochemical production in heavy metal/chemical toxin-resistant microalgae or cyanobacteria produced through the method of microevolution.

Another embodiment encompasses a novel water bioremediation system that utilizes micro-evolved microbes developed through a stepped process to capture and recover heavy metals and simultaneously produce biochemicals. Such biochemicals may be of use to the pharmaceutical industry, to the food supplement industry, to the nutraceutical industry, to the agricultural industry, to the horticultural industry, to the animal feed industry, and to the veterinary industry.

Another embodiment encompasses the remediation system disclosed herein wherein the microbes are used to produce biomass. The biomass so produced may be of use to the food industry, the animal feed industry, the pharmaceutical, the food supplement, and the nutraceutical industries (for use, for example, as excipient and/or filler).

In another embodiment, the invention provides a bioreactor, the bioreactor comprising the micro-evolved microbe, a fluid, a fluid input aperture, a fluid output aperture, and at least one gas input/output aperture. In a preferred embodiment the aperture comprises a valve. The valve may be a one-way valve, a two-way valve, or a multi-way valve.

In one preferred embodiment the fluid is wastewater. In an alternative preferred embodiment the fluid is organic waste. In a yet further alternative embodiment the fluid is a hydrocarbon mixture.

Strain Development Methods

During the microevolution process the following steps are carried out:

i) A single microalga (MA)/cyanobacterium(CB) is separated by serial dilution.

ii) A culture is grown up from this single MA/CB. All MA/CB in the culture should be clones of the single starting bacterium except for any slight genetic changes (mutations) which have occurred by chance during this period of division.

Cells were grown up (and selected after exposure) as follows: The mutations which were advantageous to survival in high metal concentration were selected for by exposure to differing concentrations of chemicals/heavy. During exposure to heavy metal most of the cells will die off as they cannot tolerate such high concentrations of heavy metal, however a single cell with a mutation which enables survival will survive and grow up/divide into a tolerant culture. We identified whether this has occurred by acclimation (change in gene-expression) or micro-evolution (change in gene) using population statistics method based on Luria-Delbruck and Poisson distribution (See “Fluctuation Analysis”, below).

When a number of resistant cultures had been identified these were tested for the concentrations of heavy metal they could tolerate. They were exposed to different concentrations and the cell numbers before and after exposure counted. Then a bell shaped curve was produced for concentration versus cell survival for each culture which showed resistant. From this an EC50 was calculated (measure of toxicity—concentration of heavy metal at which half the cells in the culture die). The test organism was then tested for uptake rate and mechanism of uptake to inform processing method for recovery.

Fluctuation Analysis

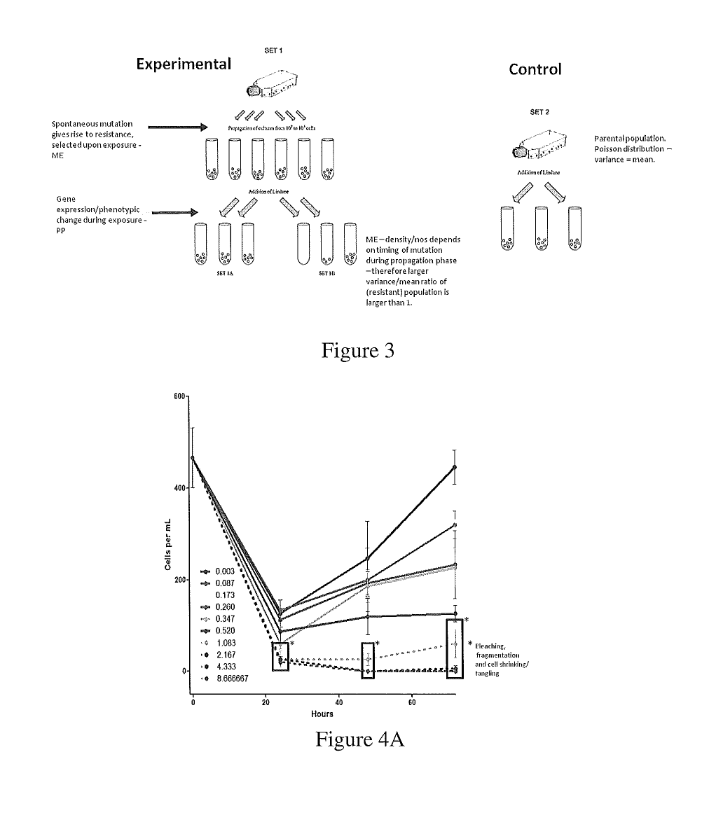

The result from the fluctuation analysis after the selected period can explain the presence of resistant cells in two ways. Each result is interpreted as the independent consequence of two different phenomena of adaptation. In the first case, if resistant cells arose during the exposure to the selective agent (that is, by physiological adaptation), the variance in the number of cells per culture would be low because every cell is likely to have the same chance of developing resistance (set 1A; FIG. 3). Consequently, inter-culture (tube-to-tube) variation would be consistent with the Poisson model (that is, the variance of these replicate samples would be equal to the mean). By contrast, if resistant cells arose before the exposure to the selective agent (that is, genetic adaptation by rare spontaneous mutation occurring during the time in which the cultures grew from N₀ to Nt), a high variation in the number of resistant cells per culture would be found (set 1B; FIG. 3). The tube-to-tube variation would not be consistent with the Poisson model (that is, variance>mean). Fluctuation analysis can likewise show the possibility of no adaptation if no resistant cells are observed after the incubation period in any of the experimental cultures. Since set 2 is the control of the experiment, variation is due only to random sampling and variation from tube to tube would be consistent with the Poisson model. If the variance/mean ratio of set 1 is significantly greater than the variance/mean ratio of set 2 (fluctuation), this confirms that resistant cells arose by rare mutations that occurred before exposure (that is, a pre-selective mechanism). If a similar variance: mean ratio between set 1 and set 2 is found, it confirms that resistant cells arose during the exposure to the selecting agent (that is, a post-selective mechanism).

A. platensis strains with resistance to 100 and 200-fold normal optimal concentration of Cu²⁺ ions were created through adaptive microevolution. Strains are now being tested for their dose-response to copper (with an ECso calculation) and compared to ‘pre-microevolved’ strains to assess the extent of resistance acquired and the optimal culture [Cu²⁺ ions] of the ‘microevolved’ strains.

Strain Stability

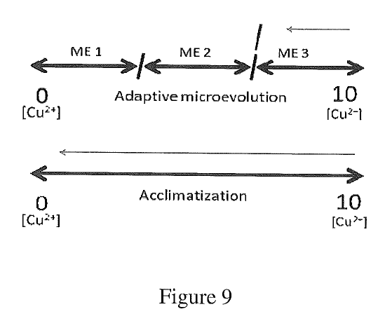

The benefit of producing a resistant strain through this ME process, ensuring the mechanism of resistance is microevolution, may produce a more stable strain for commercial use. Whereas in the acclimation process (resistance produced through increasing and prolonged exposure), resistant strains can easily revert back to optimal growth at lower concentrations (reversion shown in red arrows, FIG. 9); through adaptive microevolution, each round of microevolution produces a strain with a narrow window of resistance/optimal growth concentration, so if culturing conditions change, the strain cannot revert back to optimal growth at lower concentrations, so preserving the extent of acquired resistance.

Biorefinery Model

The combined use of high heavy metal (or toxic chemical) wastewater as a feed stock for synergistic bioremediation/heavy metal recovery and nutraceutical/biochemical production in heavy metal/chemical toxin-resistant microalgae or cyanobacteria produced through the method of microevolution.

Another example is a novel water bioremediation system that utilises micro-evolved microbes developed through a stepped process to capture and recover heavy metals and simultaneously produce biochemicals and biomass.

The invention will be more readily understood by reference to the following examples, which are included merely for purposes of illustration of certain aspects and embodiments of the present invention and not as limitations.

### Examples

### Example I

### Start-Point Selections

The cyanobacterium Arthrospira platensis (commercially known as Spirulina) was used, and resistance to copper (Cu²⁺ ions) was investigated. This particular metal was selected as a starting point for experiments as copper is a common marine pollutant and a contaminant.

### Example II

### Outline of the Experimental Process

FIG. 2 illustrates a typical outline of the micro-evolution process. The experiment was performed based on the method from Gonzalez et al., (2012) [14]. Following FIG. 2, the culture was cloned through the isolation of a single alga or cyanobacterium, to avoid the inclusion of previous spontaneous mutations, followed by growth. Cloning was performed through microdilution in microwell plates, where the culture was diluted to the culture density of 1 microbe (micro-organism, MO) per 200 μl, providing a single cell in each well (although if microalgae/cyanobacteria can grow on solid medium, isolation on medium agar can also be performed). Propagation of the cloned culture was performed from 200 μl in increasing volume flasks with foam stoppers in a constant temperature room 18° C.+/−1° C., average light intensity 2800 lux with 8:16 dark:light cycle.

10e2 MOs were then subcultured into 10 ml 40 vials (set 1), and cultures propagated further in vials and propagation flask (for set 2) (FIG. 3). Before exposure, controls ‘set 2’ were prepared through subculturing from flask into 20 vials. During the exposure stage, CuSO₄ solution was added to the 60 vials of each concentration set to make-up the final concentrations of 0.3, 0.6, 1, 2 and 4 mg/Cu²⁺ ions. Vials were stored under propagation conditions described above for a period of 60 days, with daily agitation, ensuring resistant cells could generate enough progeny to be detected. After the incubation period, fluctuation analysis was performed to identify those cultures which resistance arose through microevolution.

### Example III

### Toxicology

Toxicology assays and EC50 calculations for copper were performed on Arthrospira platensis (CCMP1295). Assessments were performed under aseptic conditions in 2 stages through an initial range-finding assay (data not shown) using CuSO₄ solutions to detect the region of the EC50, followed by a final toxicology assay over 72 hours following British Standard BS EN ISO 10253:2006 [15] with ECso determination where inhibition is measured as a reduction in specific growth rate, relative to control cultures grown under identical conditions. Visual analysis was also performed to assess the effects of metal toxicity on morphology. Statistical analysis was performed using GraphPad Prism 5 Non-linear transform log(agonist) vs. response—variable response function.

### Example IV

### A. platensis Toxicity Assay

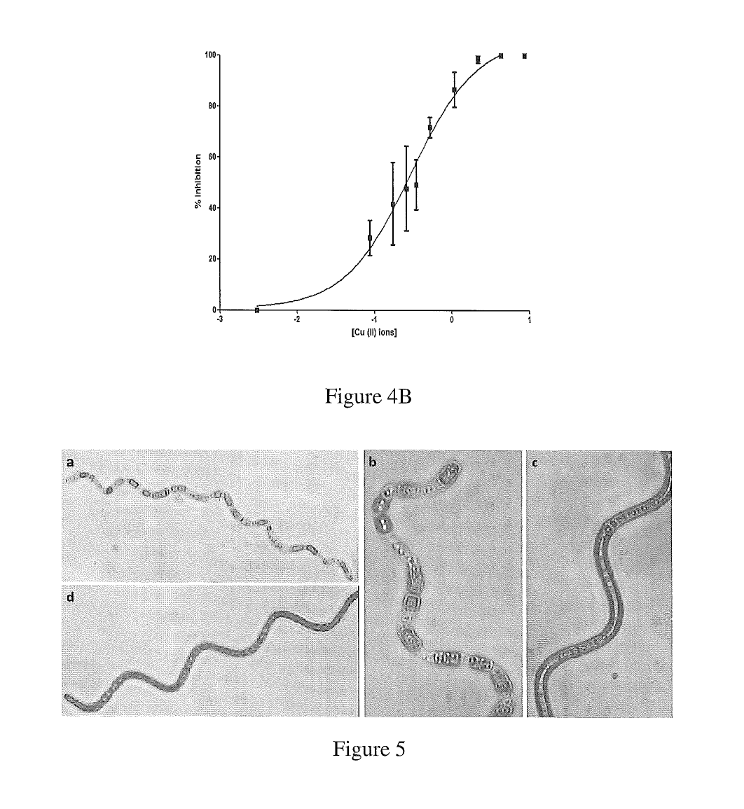

FIGS. 4A and 4B show the results of the toxicity assay using A. platensis. EC50 of Cu²⁺ ions for A. platensis was calculated at 0.2976 (in the 95% CI range of 0.17-0.5208 mg/L with an R squared value of 0.8654).

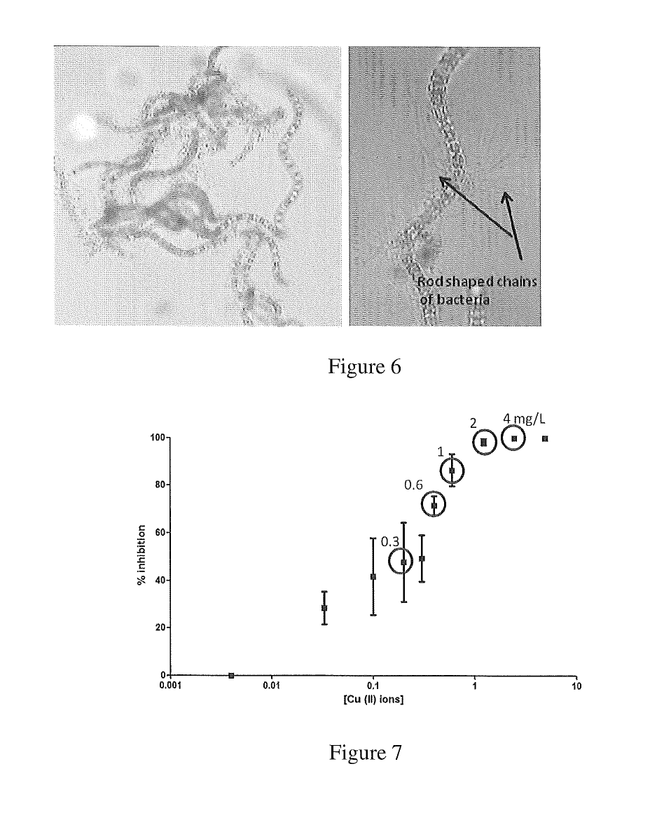

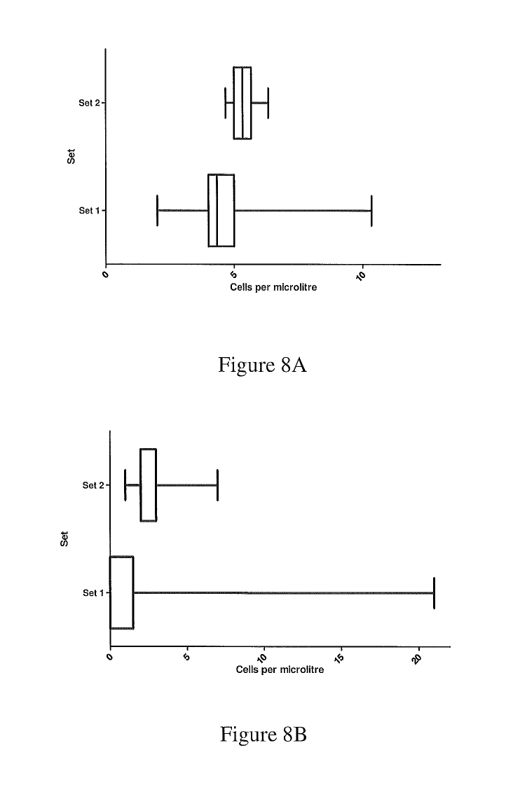

Visual observations (FIG. 5 and FIG. 6) showed filament fragmentation, bleaching and entanglement/clumping of cyanobacterium.

### Example V

### A. platensis Concentration Selections for Micro-Evolution (ME)

FIG. 7 shows the [copper] that were selected to be used for the micro-evolution process. Five copper concentrations were selected for exposure during the next step of the microevolution process (FIG. 7). These were 0.3, 0.6, 1, 2 and 4 mg/L. Concentrations in pot ale are around the concentrations of 2-6 ppm or mg/L [16], so strains produced resistant to the concentrations selected could be commercially useful. Doses for exposure are usually selected at 2-4-fold higher than those doses causing 100% inhibition. Conservatively, doses lower than 100% inhibition were selected.

### Example VI

### Post-Exposure Assessment

TABLE 1
Summary of results

| Cu²⁺ ion concentration (mg/L) | Outcome |
|---|---|
| 0.3 | RSM |
| 0.6 | RSM |
| 1 | No adaptation |
| 2 | No adaptation |
| 4 | No adaptation |

For the three higher Cu²⁺ ion concentrations 1, 2 and 4 mg/L, no cells were detected in any of the vials, demonstrating no adaptation was achieved (Table 1 and Table 2). Cells were however present in the two lower concentrations of 0.3 and 0.6 mg/L Cu²⁺ ions demonstrating resistant cells. Fluctuation analysis of the data collected for these two concentration sets showed significantly higher variance/mean ratios of set 1 compared to set 2, indicating resistance in set 1 arose by a different mechanism to normal acclimation, relating to microevolution (Table 2). The variance/mean ratio in set 2 for 0.06 mg/L Cu²⁺ ions is 1.1, conforming to the expected Poisson distribution and normal randomization of the control. However, the variance/mean ratio in set 2 for 0.03 mg/L Cu²⁺ ions is much lower than 1, indicating a more uniform than random distribution in the control for this concentration set.

TABLE 2
Fluctuation analysis from Cu²⁺ ions-sensitivity to Cu²⁺ ions-resistance
under different concentrations of Cu²⁺ ions.

| | Set1 | Set2 |
|---|---|---|
| Adaptation to 0.3 mg/L Cu²⁺ ions | | |
| No. of replicates | 40 | 20 |
| No. of cultures containing following no. [Cu] | | |
| resistant MOs | | |
| 0 | 0 | 0 |
| 2 to 4 | 22 | 0 |
| 4 to 6 | 12 | 20 |
| 6 to 8 | 4 | 0 |
| 8+ | 2 | 0 |
| VAR/MEAN ratio (no. of resistant cells per | 0.75 | 0.06 |
| replicate) | | |
| cells per microlitre | | |
| Adaptation to 0.6 mg/L Cu²⁺ ions | | |
| No. of replicates | 40 | 20 |
| No. of cultures containing following no. [Cu] | | |
| resistant MOs | | |
| 0 | 30 | 0 |
| 1 to 5 | 4 | 18 |
| 5 to 10 | 1 | 2 |
| 10 to 15 | 3 | 0 |
| 15+ | 2 | 0 |
| VAR/MEAN ratio (no. of resistant cells per | 12.18 | 1.10 |
| replicate) | | |
| cells per 50 microlitres | | |
| Adaptation to 1 mg/L Cu²⁺ ions | | |
| No. of replicates | 40 | 20 |
| No. of cultures containing following no. [Cu] | | |
| resistant MOs | | |
| 0 | 40 | 20 |
| Adaptation to 2 mg/L Cu²⁺ ions | | |
| No. of replicates | 40 | 20 |
| No. of cultures containing following no. [Cu] | | |
| resistant MOs | | |
| 0 | 40 | 20 |
| Adaptation to 4 mg/L Cu²⁺ ions | | |
| No. of replicates | 40 | 20 |
| No. of cultures containing following no. [Cu] | | |
| resistant MOs | | |
| 0 | 40 | 20 |

A. platensis strains with resistance to 100- and 200-fold normal optimal concentration of Cu²⁺ ions were created through adaptive microevolution. Strains are now being tested for their dose-response to copper (with an ECso calculation) and compared to ‘pre-microevolved’ strains to assess the extent of resistance acquired and the optimal culture [Cu²⁺ ions] of the ‘microevolved’ strains.

### REFERENCES

1. Lopez-Rodas, V. et al. 2001. Resistance of microalgae to modern water contaminants as the result of rare spontaneous mutations. European Journal of Phycology 36(2): 179-190.
2. Lopez-Rodas, V., et al. 2009. A fascinating example of microalgal adaptation to extreme crude oil contamination in a natural spill in Arroyo Minero, Rio Negro, Argentina. Anales de la Real Academia de Farmacia 75: 883-899.
3. Bradshaw, A. D., Hardwick, K. 1989. Evolution and stress—genotypic and phenotypic components. Biological Journal of the Linnean Society 37(1-2): 137-155.
4. Rai, L. C. and Gaur, J. P. 2001. Algal Adaptation to Environmental Stresses: Physiological, Biochemical and Molecular Mechanisms. Springer-Verlag, Berlin.
5. Sniegowski, P. D. 2005. Linking Mutation to Adaptation: Overcoming Stress at the spa. New Phytologist 166(2): 360-362.
6. Alexander, M. 1999. Biodegradation and Bioremediation. Second ed. Academic Press, San Diego.
7. De Philippis, R. and Vincenzini, M. 1998. Exocellular polysaccharides from cyanobacteria and their possible applications. FEMS Microbiology Reviews 22(3): 151-175.
8. Ting, Y. P., Teo, W. K., and Soh, C. Y. 1995. Gold uptake by Chlorella vulgaris. Journal of Applied Phycology 7(1): 97-100.
9. Stokes. 1983. Responses on freshwater algae to metals. In: Round F E, Chapman D H J (eds) Advances in phycological research, vol 2. Elsevier, Amsterdam 87-112
10. Silver, S. and Phung, L. T. 1996. Bacterial heavy metal resistance: new surprises. Annu Rev Microbiol 50: 753-89.
11. Rauser, W. E. 1990. Phytochelatins. Annual Review of Biochemistry 59: 61-86.
12. Steffens, J. C. 1990. The Heavy Metal-Binding Peptides of Plants. Annual Review of Plant Physiology and Plant Molecular Biology 41: 553-575.
13. Rai, L. C., Gaur, J. P., Kumar, H. D. 1981. Protective effects of certain environmental factors on the toxicity of zinc, mercury, and methylmercury to Chlorella vulgaris Environ Res 25: 250-259.
14. Gonzalez, R. et al. 2011. Adaptation of microalgae to lindane: A new approach for bioremediation. Aquatic Toxicology 109: 25-32.
15. 2006. Water quality. Marine algal growth inhibition test with Skeletonema costatum and Phaeodactylum tricornutum, British Standard BS EN ISO 10253:2006.
16. Mohana, S., Acharya, B. K., Madamwar, D. 2009. Distillery spent wash: Treatment technologies and potential applications. Journal of Hazardous Material 163: 12-25.

1. A remediation system, the remediation system comprising a vessel and a microbial population having tolerance to a test composition, wherein the system is selected from the group consisting of a bioremediation and recovery system, a carbon capture system, a biochemical production system and a biomass production system.

2. The remediation system of claim 1, wherein the microbial population is cultured from a microbe selected using a micro-evolution process for creating a microbial population tolerant to a test composition, the micro-evolution process comprising the following steps: (i) providing a single microbe, the microbe isolated by serial dilution of a microbial population; (ii) providing a culture medium; (iii) adding the single microbe to the culture medium; (iv) providing the single microbe in the culture medium an environment whereby the single microbe replicates thereby creating daughter microbes; (v) treating the culture medium with a test composition whereby the test composition selects those daughter microbes having genetic resistance to the test composition; (vi) using fluctuation analysis to determine whether the selected daughter microbes comprise a change in gene expression (transcription and/or translation) or a change in a gene (mutation and/or chemical modification); (vii) selecting only those daughter microbes having a change in a gene, wherein the change in a gene is micro-evolution; the micro-evolution process resulting in a microbial population tolerant to the test composition.

3. The remediation system of claim 2 wherein the microbe is a microalga.

4. The remediation system of claim 3 wherein the microalga is selected from the group consisting of Chlamydomonas, Chlorella, and Scenedesmus.

5. The remediation system of claim 4 wherein the microalga is Chlamydomonas nivalis.

6. The remediation system of claim 4 wherein the microalga is Chlorella vulagris.

7. The remediation system of claim 4 wherein the microalga is Scenedesmus dimorphus.

8. The remediation system of claim 2 wherein the microbe is a cyanobacterium.

9. The remediation system of claim 8 wherein the cyanobacterium is Arthrospira platensis.

10. The remediation system of claim 3 wherein the microbe is a gram-positive bacterium.

11. The remediation system of claim 10 wherein the gram-positive bacterium is Bacillus subtilis.

12. The remediation system of claim 2 wherein the test composition comprises a heavy metal selected from the group consisting of arsenic, cadmium, hexavalent chromium, lead, mercury, antimony, cobalt, copper, iron, manganese, molybdenum, nickel, selenium, silver, tin, vanadium, and zinc.

13. The remediation system of claim 2 wherein the test composition comprises an organic compound selected from the group consisting of organophosphates, organochlorines, carbamates, polycyclic aromatic hydrocarbons, polychlorinated biphenyls, triazines, phenoxy herbicides, dioxins, and alkaloids.

14. The remediation system of claim 2 where the test composition is a metal selected from the group consisting of aluminium, beryllium, and magnesium.

15. The remediation system of claim 2, further comprising subjecting the microbe to an environmental stress but not to the test composition.

16. The remediation system of claim 15, wherein the environmental stress is selected from the group consisting of a rapid change in temperature, change in alkalinity, change in dissolved carbon dioxide, change in dissolved oxygen, change in salinity, change in nutrient levels of the medium, change in the incident wavelength of light, and change in NH₄O₃ levels.

17. The remediation system of claim 2, wherein the vessel comprises a fluid, a fluid input aperture, a fluid output aperture, and at least one gas input/output aperture.

18. The remediation system of claim 17, wherein the aperture comprises a valve, wherein the valve is selected from the group consisting of a one-way valve, a two-way valve, and a multi-way valve.

19. The remediation system of claim 17, wherein the fluid is selected from the group consisting of wastewater, organic waste, and a hydrocarbon mixture.

20. A carbon-capture process using the remediation system of claim 1, wherein further the microbial population fixes carbon dioxide from the environment and metabolically sequesters the carbon dioxide into a microbial biochemical.

21. The carbon-capture process of claim 20, wherein the microbial biochemical further is harvested and isolated from the microbial population.

22-25. (canceled)

26. A method for remediating a pollutant, the pollutant comprising a test composition, the method comprising the steps of: (i) providing a single microbe, the microbe isolated by serial dilution of a microbial population; (ii) providing a culture medium; (iii) adding the single microbe to the culture medium; (iv) providing the single microbe in the culture medium an environment whereby the single microbe replicates thereby creating daughter microbes; (v) treating the culture medium with a test composition whereby the test composition selects those daughter microbes having genetic resistance to the test composition; (vi) using fluctuation analysis to determine whether the selected daughter microbes comprise a change in gene expression (transcription and/or translation) or a change in a gene (mutation and/or chemical modification); (vii) selecting only those daughter microbes having a change in a gene, wherein the change in a gene is micro-evolution; the micro-evolution process resulting in a microbial population tolerant to the test composition; (viii) culturing the microbial population with the pollutant for a predetermined period of time; (ix) separating the microbial population from the pollutant, the method resulting in the remediation of the pollutant.

27. The method of claim 26, wherein the method for remediating the pollutant results in remediation.

28. The method of claim 27, wherein the remediation is bioremediation.

29. The method of claim 28, wherein the bioremediation is selected from the group consisting of wastewater bioremediation, contaminated water bioremediation, hazardous waste bioremediation, and radioactive waste bioremediation.

30. The remediation system of claim 1, wherein the remediation system is selected from the group consisting of a carbon-capture system, a wastewater treatment system, a biomass production system, and a biochemical production system.
